(19) United States
(12) Patent Application Publication
(10) Pub. No.: US 2025/0152026 A1
(43) Pub. Date: May 15, 2025

(54) PRESSURE CUFF OVERTIGHTENING DETECTION ALGORITHM

(71) Applicant: Becton, Dickinson and Company, Franklin Lakes, NJ (US)

(72) Inventors: Yasser Khaled Morsy, Bridgeville, PA (US); Zhongping Jian, Irvine, CA (US); Boris Reuderink, Amersfoort (NL); Hans Jean Paul Kuijkens, Amstelveen (NL); Jeroen Van Goudoever, Amsterdam (NL); George Hua Ma, Irvine, CA (US)

(21) Appl. No.: 19/019,434

(22) Filed: Jan. 13, 2025

Related U.S. Application Data
(63) Continuation of application No. PCT/US2023/027280, filed on Jul. 10, 2023.
(60) Provisional application No. 63/389,338, filed on Jul. 14, 2022.

(51) Int. Cl. A61B 5/022; A61B 5/00; A61B 5/0235

(52) U.S. Cl. CPC A61B 5/02241; A61B 5/0235; A61B 5/746

(57) ABSTRACT

A system includes a hardware unit that stores signal distortion detection code. The code causes the system to execute steps of a method for evaluating the fit and placement of a hemodynamic sensor. The method includes obtaining hemodynamic data from the hemodynamic sensor and determining an arterial pressure waveform based on the hemodynamic data. A distortion score is determined based on one or more features extracted from the arterial pressure waveform and a weighting module. The method includes selectively invoking a sensory alarm based on a comparison of the distortion score to a predetermined criterion or predetermined criteria.

### CROSS-REFERENCE TO RELATED APPLICATION(S)

This application is a continuation of PCT Application No. PCT/US2023/027280, filed Jul. 10, 2023, and entitled “PRESSURE CUFF OVERTIGHTENING DETECTION ALGORITHM,” which in turn claims the benefit of U.S. Provisional Application No. 63/389,338, filed Jul. 14, 2022, and entitled “PRESSURE CUFF OVERTIGHTENING DETECTION ALGORITHM,” the disclosures of which are hereby incorporated by reference in their entireties.

### BACKGROUND

The present disclosure relates generally to arterial pressure monitoring, and more specifically to evaluating fit and/or location quality of hemodynamic sensor through analysis of the arterial pressure signal.

Volume clamping is a technique for non-invasively measuring arterial pressure in which pressure is applied to a patient's finger in such a manner that arterial pressure may be balanced by a time varying pressure to maintain a constant arterial volume. In a properly fitted and calibrated system, the applied time varying pressure is equal to the arterial pressure in the finger. The applied time varying pressure may be measured to provide a reading of the patient's arterial pressure.

This may be accomplished by a finger cuff that is arranged around a finger of a patient. The finger cuff may include an infrared light source, an infrared sensor, and an inflatable bladder. The infrared light may be sent through the finger in which a finger artery is present. The infrared sensor picks up the infrared light and the amount of infrared light registered by the sensor may be inversely proportional to the artery diameter and indicative of the pressure in the artery.

In the finger cuff implementation, by inflating the bladder in the finger cuff, a pressure is exerted on the finger artery. If the pressure is high enough, it will compress the artery and the amount of light registered by the sensor will increase. The amount of pressure necessary in the inflatable bladder to compress the artery is dependent on the blood pressure. By controlling the pressure of the inflatable bladder such that the diameter of the finger artery is kept constant, the blood pressure may be monitored in very precise detail as the pressure in the inflatable bladder is directly linked to the blood pressure. In a typical present day finger cuff implementation, a volume clamp system is used with the finger cuff. The volume clamp system typically includes a pressure generating system and a regulating system that includes: a pump, a valve, and a pressure sensor in a closed loop feedback system that are used in the measurement of the arterial volume. To accurately measure blood pressure, the feedback loop provides sufficient pressure generating and releasing capabilities to match the pressure oscillations of the subject's blood pressure.

Current finger cuffs are easy to place on a patient's finger. However, it is important that the finger cuff is placed on the patient's finger correctly in order for the blood pressure measurement system to obtain and report correct blood pressure measurement values. In order to obtain a correct attachment of the finger cuff to the finger, the finger cuff needs to be placed on the finger at the correct depth, correct angle, and with the correct tightness. Current finger cuffs are often made from a flexible material that a healthcare provider wraps around the patient's finger and locks into place by simple attachment mechanisms, such as, Velcro. These current types of finger cuffs require that the healthcare provider is able to control all three of these variables (e.g., correct depth, correct angle, and correct tightness), simultaneously, while attaching the finger cuff. Unfortunately, this may result in erroneous placement and tightness of the finger cuff. This erroneous placement and tightness may result in an unsuccessful reading of the patient's blood pressure. Even where the healthcare work initially places and tightens the finger cuff correctly, the finger cuff can become incorrectly placed or tightened by patient movement or a change in the patient's condition. For example, the patient's extremities may develop edema. Swelling as a result of edema or other cause may also result in an unsuccessful reading of the patient's blood pressure.

### SUMMARY

A system for monitoring the fit of a hemodynamic sensor (e.g., a finger cuff) to a patient, in accordance with an exemplary example of this disclosure, includes a hardware unit. The hardware unit includes a hardware processor, an analog-to-digital converter (ADC), and a system memory. The system further includes a signal distortion detection code and weighting module stored in the system memory and a sensory alarm. The hardware processor is configured to execute the signal distortion detection code to obtain hemodynamic data, which may be converted by the ADC, from a signal received from the hemodynamic sensor on an ongoing basis. The code is further configured to obtain an arterial pressure waveform based on the hemodynamic data and extract a plurality of features from the first arterial pressure waveform indicative of distortion of the first arterial pressure waveform associated with a fit or location quality of hemodynamic sensor to the patient. Using the weighting module and the extracted features (i.e., distortion parameters), the code is further configured to determine a distortion score and invoke a sensor alarm if the distortion score satisfies a predetermined criterion.

### BRIEF DESCRIPTION OF THE DRAWINGS

FIG. 1 is a schematic of an exemplary patient monitoring system.

FIG. 2 is a schematic of an exemplary patient monitoring system that includes a finger cuff.

FIG. 3A is a schematic of a finger cuff and a pressure controller of an exemplary hemodynamic sensor.

FIG. 3B is a schematic of a finger cuff in an uninstalled state.

FIG. 4A, FIG. 4B, and FIG. 4C are exemplary charts comparing arterial pressure parameters of a finger cuff with a normal fit to arterial pressure parameters of a finger cuff with an overtightened fit.

FIG. 5 is a schematic depicting features of an arterial pressure waveform taken a patient's finger along with associated brachial and radial arterial pressure waveforms derived from the arterial pressure waveform.

FIG. 6 is a chart depicting exemplary features of the radial arterial pressure waveform.

FIG. 7 is a chart depicting exemplary features of the brachial arterial pressure waveform.

FIG. 8 is a flow chart illustrating steps of the signal distortion detection method.

### DETAILED DESCRIPTION

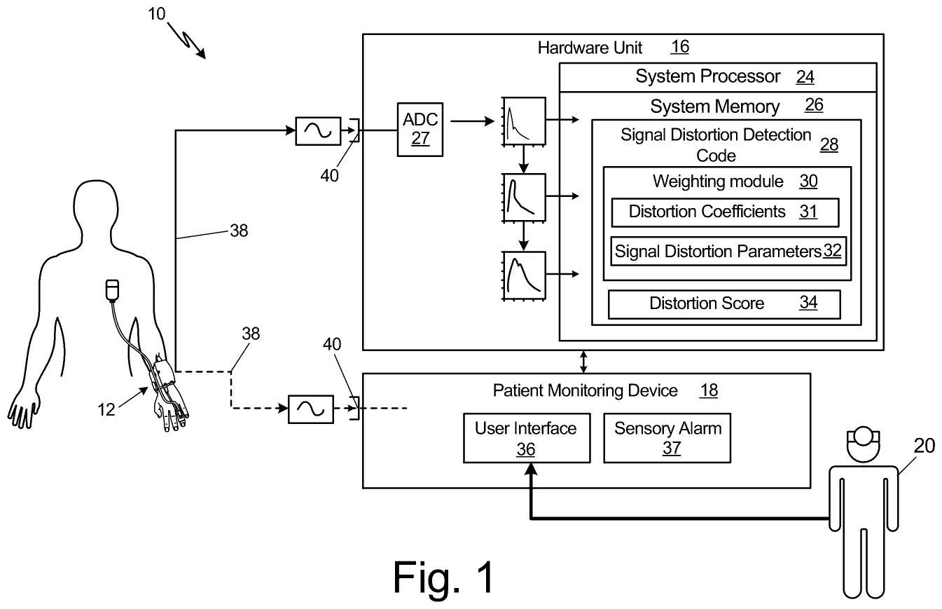

FIG. 1 is a schematic depicting components of an exemplary patient monitoring system capable of evaluating the fit and/or placement of sensors attached to a patient. As shown, patient monitoring system 10 includes hemodynamic sensor 12 fit to patient 14, hardware unit 16, and patient monitoring device 18.

Hemodynamic sensor 12 can be a non-invasive sensor fitted about an appendage of patient 14 and, in some instances, at a specific location along the patient's appendage. Misfitting or mislocating hemodynamic sensor 12 may occur incidentally during application by practitioner 20 or may develop from patient movement and/or changes in the patient's condition (e.g., swelling of the patient's appendage). When misfitted and/or mislocated, hemodynamic sensor 12 may provide erroneous hemodynamic data to hardware unit 16, impairing monitoring of patient 14.

Hardware unit 16 includes system processor 24, system memory 26, and analog-to-digital (ADC) converter 27, which interface with patient monitoring device 18. In some instances, hardware unit 16 is discrete from patient monitoring device 18, which may be installable into a corresponding receptacle or slot of patient monitoring device 18. In other examples, hardware unit 16 integrates with internal components of patient monitoring device 18. In further examples, one or more components and/or described functionality can be distributed among multiple hardware units 16, patient monitoring devices 18, and/or other patient monitoring equipment.

System processor 24 executes signal distortion detection code 28, which uses weighting module 30 and signal distortion parameters 32 to determine distortion score 34 representing fit quality and/or position quality of hemodynamic sensor 12. Examples of system processor 24 can include any one or more of a microprocessor, a controller, a digital signal processor (DSP), an application specific integrated circuit (ASIC), a field-programmable gate array (FPGA), or other equivalent discrete or integrated logic circuitry.

System memory 26 can be configured to store information within hardware unit 16 during operation. As illustrated in FIG. 1, system memory 26 stores signal distortion detection code 28. System memory 26, in some examples, is described as computer-readable storage media. In some examples, a computer-readable storage medium can include a non-transitory medium. The term “non-transitory” can indicate that the storage medium is not embodied in a carrier wave or a propagated signal. In certain examples, a non-transitory storage medium can store data that can, over time, change (e.g., in RAM or cache). System memory 26 can include volatile and non-volatile computer-readable memories. Examples of volatile memories can include random access memories (RAM), dynamic random-access memories (DRAM), static random-access memories (SRAM), and other forms of volatile memories. Examples of non-volatile memories can include, e.g., magnetic hard discs, optical discs, flash memories, or forms of electrically programmable memories (EPROM) or electrically erasable and programmable (EEPROM) memories.

Hardware unit 16 receives hemodynamic data from hemodynamic sensor 12. Hemodynamic data can include any one or more features of an arterial pressure waveform or derived from an arterial pressure waveform. The arterial pressure waveform can be measured at a radial location, a brachial location, or a finger location of patient 14. Further, features from multiple waveforms (e.g., from one or more arterial waveforms taken at a radial location, a brachial location, and/or a finger location) can be used in combination by signal distortion detection code 28. For example, a non-exhaustive list of potential arterial pressure waveform features includes mean arterial pressure, maximum arterial pressure, minimum arterial pressure, pulse rate, pulse pressure, maximum and/or minimum rate of pressure change with respect to time during a heartbeat (or a subset of heartbeats), maximum and/or minimum rate of pressure change with respect of time during the systolic phase and/or diastolic phase of a heartbeat (or a subset of heartbeats), end diastolic pressure, diastolic gradient, systolic pressure gradient, pulse transit time, cardiac output, stroke volume, blood temperature, systolic rise area, diastolic average pressure, systemic vascular resistance, exponential constant associated with systolic decay, among other possible parameters.

Weighting module 30 applies distortion coefficients 31, determined via training, to signal distortion parameters 32, which are features extracted from an arterial pressure waveform. Certain signal distortion parameters 32 may be indicative of hemodynamic sensor 12 with a proper fit and/or proper location while other signal distortion parameters 32 may be indicative of hemodynamic sensor 12 with an improper fit and/or improper location. Based on distortion coefficients 31 applied by weighting module 30 and signal distortion parameters 32, signal distortion detection code 28 determines distortion score 34 representative of the fit quality and/or location quality of hemodynamic sensor 12.

The selection of distortion coefficients 31 and/or signal distortion parameters 32 can be accomplished via training (e.g., offline training) of the distortion detection model using machine learning or other techniques to minimize a cost function representing the error of the distortion detection model output to the true value of training subsets that define properly fit and improper fit hemodynamic sensors 12. Training of the distortion detection model can be accomplished using a variety of supervised and/or unsupervised techniques known in the art. One exemplary technique includes obtaining arterial pressure feature datasets from hemodynamic sensors 12 known to have a proper fit and proper location and from different hemodynamic sensors 12 known to have an improper fit and/or improper location. Based on features associated with two or more datasets, logistic regression can be used to identify which features are indicative of proper fit and/or proper location of hemodynamic sensors 12 and which features are indicative of improper fit and/or improper location of hemodynamic sensors 12. Using this feature subset, further training of the distortion model determines weighting factors (i.e., distortion coefficients 31) for weighting module 30. The subset of features extracted from the arterial pressure waveform and indicative of a properly fit and/or improperly fit hemodynamic sensor 12 become distortion signal parameters 32.

Distortion score 34 can be a normalized value between 0 and 1 (or between 0 and 100, or other normalized ranges) with, in some examples, a higher value representing a higher likelihood that hemodynamic sensor 12 is misfit or mispositioned and a lower value representing a lower likelihood that hemodynamic sensor 12 is misfit or mispositioned. In another example, the normalized range of the distortion scores can be subdivided into two or more continuous and sequential subranges, each subrange indicative of the fit quality and/or location quality of hemodynamic sensor 12 to patient 14. The first of the two subranges (e.g., a distortion score value between 0 and 50 within a normalized range from 0 to 100) can be indicative of a properly fit hemodynamic sensor 12. The second subrange (e.g., a distortion score between 51 and 100 within a normalized range from 0 to 100) can indicate improperly fit or mislocated hemodynamic sensor 12. When the normalized range consists of two subranges, system 10 can provide a binary indication of a properly fit/location or improperly fit/located hemodynamic sensor 12. In other examples, the normalized range of distortion scores 34 can be subdivided into at least three continuous and sequential subranges. The first of the three subranges (e.g., a distortion score value between 0 and 30 within a normalized range from 0 to 100) can be indicative of a properly fit hemodynamic sensor 12. The second subrange (e.g., a distortion score between 31 and 60 within a normalized range from 0 to 100) can indicate a potential improper fit and/or potential mislocated hemodynamic sensor 12. The third subrange (e.g., a distortion score between 61 and 100 within a normalized range from 0 to 100) can indicate improperly fit or mislocated hemodynamic sensor 12. In this way, the second subrange (i.e., intermediate subrange) may provide an indication that hemodynamic sensor 12 should be refit or repositioned whereas the third subrange may indicate that hemodynamic sensor 12 should be replaced.

One example equation for determining distortion score 34 is as follows:

$$Distortion Score = \frac{100}{1 + {e}^{- ( {w}_{0} + \sum {w}_{i} {x}_{i} )}} \qquad Equation 1$$

for i=1, 2, 3, . . . , 13 (i.e., a total number of distortion parameters in the distortion detection model model)
where, xᵢ's are the example parameters determined from hemodynamic data measured by hemodynamic sensor 12, wᵢ's are the corresponding parameter weights (i.e., coefficients), and w₀ is a bias term.
x₁=mean arterial pressure
x₂=maximum arterial pressure
x₃=minimum arterial pressure
x₄=pulse rate
x₅=pulse pressure
x₆=maximum rate of pressure change with respect to time during a heartbeat (or a subset of heartbeats),
x₇=minimum rate of pressure change with respect to time during a heartbeat (or a subset of heartbeats)
x₈=maximum rate of pressure change with respect of time during the systolic phase of a heartbeat (or a subset of heartbeats)
x₉=minimum rate of pressure change with respect of time during the systolic phase of a heartbeat (or a subset of heartbeats)
x₁₀=maximum rate of pressure change with respect of time during the diastolic phase of a heartbeat (or a subset of heartbeats)
x₁₁=minimum rate of pressure change with respect of time during the diastolic phase of a heartbeat (or a subset of heartbeats)
x₁₂=end diastolic pressure
x₁₃=diastolic gradient
x₁₄=systolic pressure gradient
x₁₅=pulse transit time
x₁₆=cardiac output
x₁₇=stroke volume
x₁₈=blood temperature
x₁₉=systolic rise area
x₂₀=diastolic average pressure
x₂₁=systemic vascular resistance
x₂₂=exponential constant associated with systolic decay

Distortion score 34 can be determined using one, a selected number, or all of distortion parameters 32, and some examples of determining distortion score 34 can include the use of other parameters from multiple arterial pressure waveforms associated with different measurement locations (e.g., a radial location, a brachial location, and/or a finger location).

Patient monitoring device 18 may be any type of medical electronic device that may read, collect, process, display, etc., physiological readings/data of a patient including blood pressure, as well as any other suitable physiological patient readings. Patient monitoring device 18 can include user interface 36 and sensory alarm 37. In some examples, power/data cable 38 may connect to hardware unit 16 via one or more input and/or output connectors 40 as shown with a solid line in FIG. 1. In other examples, patient monitoring device 18 can be connected to hemodynamic sensor 12 via power/data cable 38 and one or more input and/or output connectors 40 as indicated by a dashed line in FIG. 1. In this example, hardware unit 16 receives hemodynamic data via patient monitoring device 18.

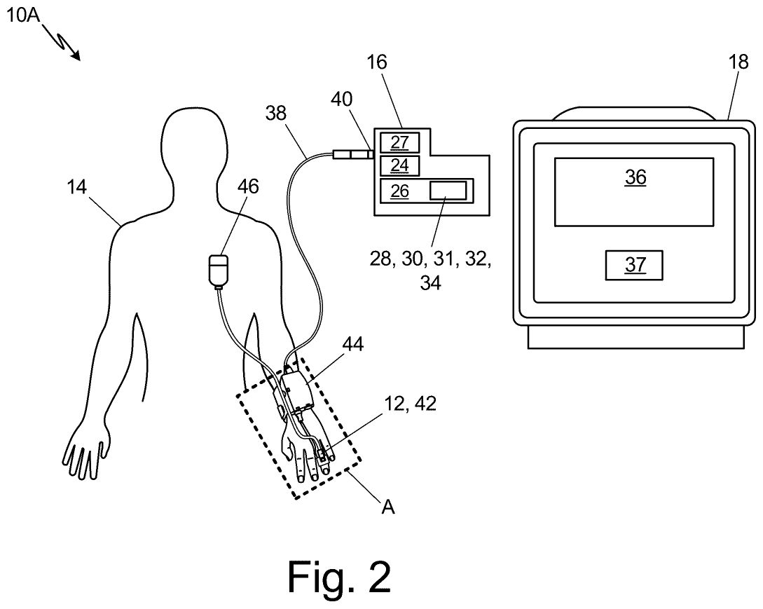

User interface 36 can be a liquid crystal display (LCD), a light-emitting diode (LED) display, an organic light-emitting diode (OLED) display, or other display device suitable for providing information to users in graphical form. In some examples, such as the example of FIG. 2, user interface 36 can be a touch-sensitive and/or presence-sensitive display device configured to receive user input in the form of gestures, such as touch gestures, scroll gestures, zoom gestures, swipe gestures, or other gesture input.

Power/data cable 38 may transmit data to and from patient monitoring device 18 and may provide power from the patient monitoring device 18 to hemodynamic sensor 12 and/or other components associated with the operation of hemodynamic sensor 12. In some examples, patient monitoring device 18 can be a hemodynamic monitor. Input and/or output (I/O) connectors 40 are configured for wired connection (e.g., electrical and/or communicative connection) with one or more peripheral components, such as one or more hemodynamic sensors

In operation, hardware unit 16 receives hemodynamic data from patient 14 via hemodynamic sensor 12 that can be digitally converted by analog-to-digital converter (ADC) 27. In response to receiving hemodynamic data of patient 14, hardware unit 16 executes signal distortion detection code 28 to determine distortion score 34 representing the fit quality and/or location quality of hemodynamic sensor 12 to patient 14. Based on distortion score 34, signal distortion detection code 28 causes patient monitoring device 18 to provide an indication of fit quality and/or location quality via user interface 36 and/or sensory alarm 37. The indication of fit quality and/or location quality of hemodynamic sensor 12 can include displaying distortion score 34 directly via user interface 36. In other examples, the indication of fit quality and/or location quality may include displaying a message or warning representative of the fit quality and/or location quality of hemodynamic sensor 12. Additionally, hardware unit 16 can invoke sensory alarm 37, such as an audible alarm, a haptic alarm, or other sensory alarm in response to determining that distortion score satisfies predetermined criteria or criterion (e.g., a distortion score within one of the predefined subranges). Accordingly, hardware unit 16 can provide a warning or message to patient monitoring device 18 so that practitioner 20 can correct improper fit and/or improper position of hemodynamic sensor 12 and/or replace hemodynamic sensor 12.

FIG. 2 is a schematic of exemplary arterial pressure monitoring system 10A. Arterial pressure monitoring system 10A is an exemplary implementation of patient monitoring system 10 shown in FIG. 1 and, except for noted differences, includes the components of patient monitoring system 10. Accordingly, arterial pressure monitoring system 10A includes hardware unit 16 and patient monitoring device 18. As shown, hardware unit 16 is a discrete module that can be inserted into a slot of patient monitoring device 18. Hardware unit 16 includes system processor 24 for executing signal distortion detection code 28 and system memory 26 for storing signal distortion detection code 28 and weighting module 30 for determining distortion score 34 based on distortion coefficients 31 and distortion parameters 32. Hardware unit additionally includes analog-to-digital (ADC) converter 27. Patient monitoring device 18 includes user interface 36 and sensory alarm 37.

Hemodynamic sensor 12 takes the form of finger cuff 42, pressure controller 44, and heart reference sensor 46. In one example, finger cuff 42 may be attached to the finger (or toe) of patient 14, and pressure controller 44 can be attached to the body of patient 14 with an attachment bracelet that wraps around the patient's wrist or hand. It should be appreciated that this is just one example of attaching pressure controller 44 and that any suitable way of attaching pressure controller 44 to a patient's body or in close proximity to a patient's body may be utilized.

Pressure controller 44 can be further connected to hardware unit 16 via power/data cable 38, which connects to hardware unit 16 via input/output connector 40. Finger cuff 42 provides hemodynamic data to hardware unit 16 representative of arterial pressure of arteries within a finger (or toe) of patient 14. Heart reference sensor 46 connects to pressure controller 44 to provide a signal representative of differential pressure between the patient's finger and heart elevation. This differential pressure signal enables pressure controller 44 to compensate hemodynamic data for changes in arterial pressure due to the patient's finger having a different elevation than the patient's heart. Together, finger cuff 42, pressure controller 44, and heart reference sensor 46 provide arterial waveform of patient 14 to hardware unit 16.

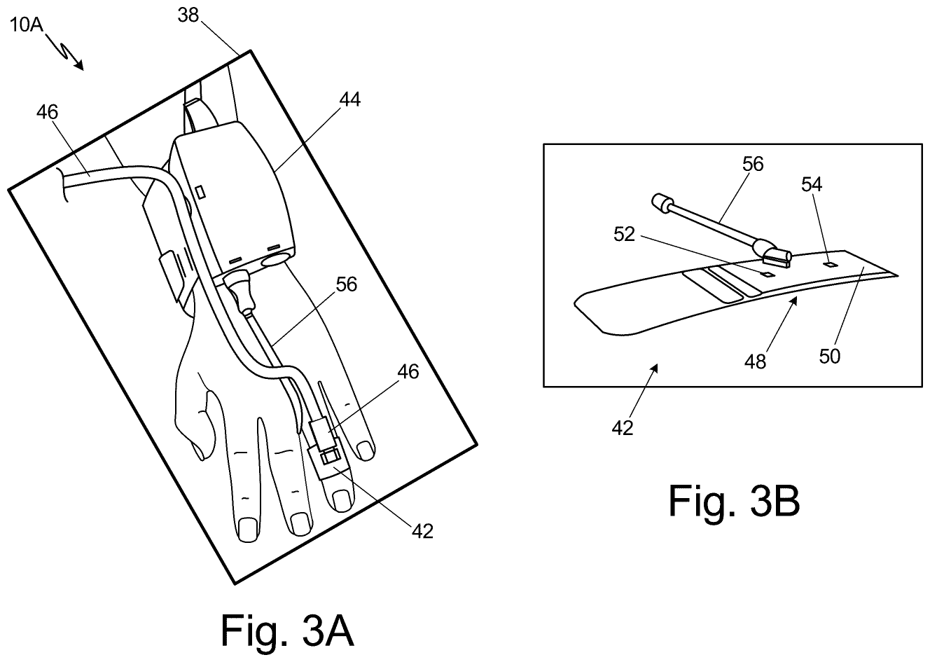

FIG. 3A is a schematic of finger cuff 42 and pressure controller 44 of exemplary hemodynamic sensor 12, which is an enlarged view of region A in FIG. 2. FIG. 3B depicts finger cuff 42 in an uninstalled state to illustrate features of finger cuff 42 hidden when fit to patient 14.

As shown in FIG. 3B, finger cuff 42 includes substrate 48, inflatable bladder 50, light-emitting diode (LED) 52, photodiode (PD) 54, and tube 56. Substrate 48 is a flexible or semi-rigid material that forms an exterior layer of finger cuff 42. Inflatable bladder 50 mounts to the interior surface of substrate 48 that is fluidly connected to tube 56. LED 52 is spaced from PD 54 along a longitudinal direction of substrate 48 and each of LED 52 and PD 54 is mounted to an interior surface of substrate 48.

Referring to FIG. 3A, pressure controller 44 can include a small internal pump, a small control valve, a pressure sensor, and control circuitry. The pump can be disposed along an internal conduit connecting tube 56 of finger cuff 42 to an intake port of pressure controller 44 that communicates with an ambient environment. The control circuitry can be configured to control the pneumatic pressure applied by the internal pump to the bladder of the finger cuff 42 to replicate the patient's blood pressure based upon measuring the signal received from the LED-PD pair of finger cuff 42. One end of heart reference sensor 46 attaches to the patient adjacent finger cuff 42 as shown in FIG. 3A while an opposite end of heart reference sensor 46 attaches to the patient's body at heart-level as shown in FIG. 2. Further, the control circuitry may be configured to control the opening of the control valve to release pneumatic pressure from the bladder. Alternatively, the control valve can be replaced with an orifice that is not controlled. In these examples, bladder pressure within finger cuff 42 varies based on operational speed or periodic operation of the internal pump and/or position of the control valve.

Continuing with this example, a patient's hand may be placed beside the patient's body in a sitting or a prone position for measuring a patient's blood pressure with the blood pressure measurement system 10. Pressure controller 44 of system 10A may be coupled to bladder 50 of the finger cuff 42 in order to provide pneumatic pressure to bladder 50 for use in blood pressure measurement. Pressure controller 44 may be coupled to hardware unit 16 (shown in FIGS. 1-2) through power/data cable 38. Accordingly, pressure controller 44 operates to vary an internal pressure of bladder 50 according to the volume clamp method, previously described, and outputs a signal representative of the arterial pressure within a finger (or toe) of patient 14 as a function of time (i.e., an arterial pressure waveform).

The arterial pressure waveform, which initially represents the arterial pressure within the patient's finger, can be mathematically transformed to represent arterial pressure waveforms at location such as a brachial site and/or a radial site of patient 14. As used herein, such radial arterial pressure waveforms and brachial arterial pressure waveforms are reconstructed from the finger arterial pressure waveform rather than measured directly (e.g., via an invasive catheterization technique).

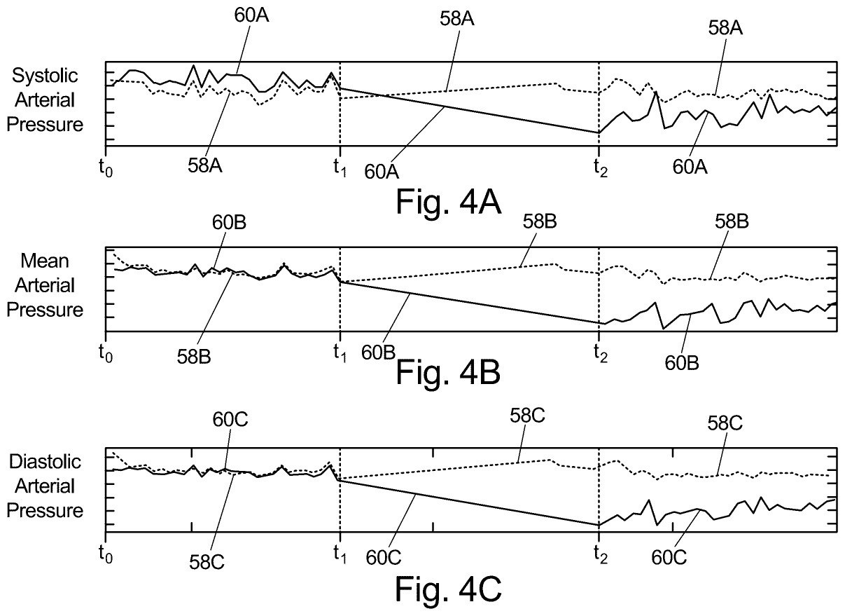

FIG. 4A, FIG. 4B, and FIG. 4C are charts depicting exemplary arterial pressure features representative of a radial site within patient 14 and measured using finger cuff 42, pressure controller 44, and heart reference sensor 46 (shown in FIG. 2). As shown in each of FIG. 4A, FIG. 4B, and FIG. 4C, an arterial pressure feature from a properly fit finger cuff 42 and from an improperly fit finger cuff 42 are compared. Based on this comparison, arterial pressure features with the tightest correlation to a properly fit and/or improperly fit finger cuff 42 can be identified and used to determine distortion score 34 via Equation 1. Repeating this process for each potential arterial pressure waveform feature may identify a set of arterial pressure waveform features indicative of a properly fit finger cuff 42 and/or an improperly fit finger cuff 42.

Referring to FIGS. 4A, 4B, and 4C, arterial parameters 58A, 58B, and 58C are indicative of systolic arterial pressure, mean arterial pressure, and diastolic arterial pressure, respectively, of a properly fitted and located first finger cuff. Arterial pressure parameters 60A, 60B, and 60C are representative of systolic arterial pressure, mean arterial pressure, and diastolic arterial pressure, respectively, produced by a second finger cuff. Prior to time step t₁, both first and second finger cuffs have proper tightness. The measurement of the first finger cuff and the second finger cuff were both stopped at time step t₁, the second cuff was then intentionally overtightened, and the measurement continues at time t₂. The first finger cuff and the second finger cuff are each installed on separate fingers of the same patient 14 in order to compare arterial pressure output from each figure cuff. As shown in FIG. 4A, FIG. 4B, and FIG. 4C, overtightening the second finger cuff at time t₂ causes arterial parameters 60A, 60B, and 60C to deviate relative to a properly fit and located first finger cuff. In this particular example, arterial parameters 60A, 60B, and 60C decrease relative to corresponding parameters produced by a properly fit first finger cuff (e.g., arterial parameters 58A, 58B, and 58C). Local maximum pressures and/or local minimum pressures of parameters 60A, 60B, and 60C relative to mean values of respective parameters 60A, 60B, and 60C are greater than or less than corresponding local maximum pressures and local minimum pressures of respective parameters produced by the first finger cuff (e.g., arterial parameters 58A, 58B, and 58C). Accordingly, these deviations can be used to identify arterial pressure features indicative of a proper fit and/or improper fit of finger cuff 42. Once identified, the deviation of the arterial pressure features relative to a properly fit finger cuff 42 characterize the effects of improperly fitting finger cuff 42 to a patient's finger.

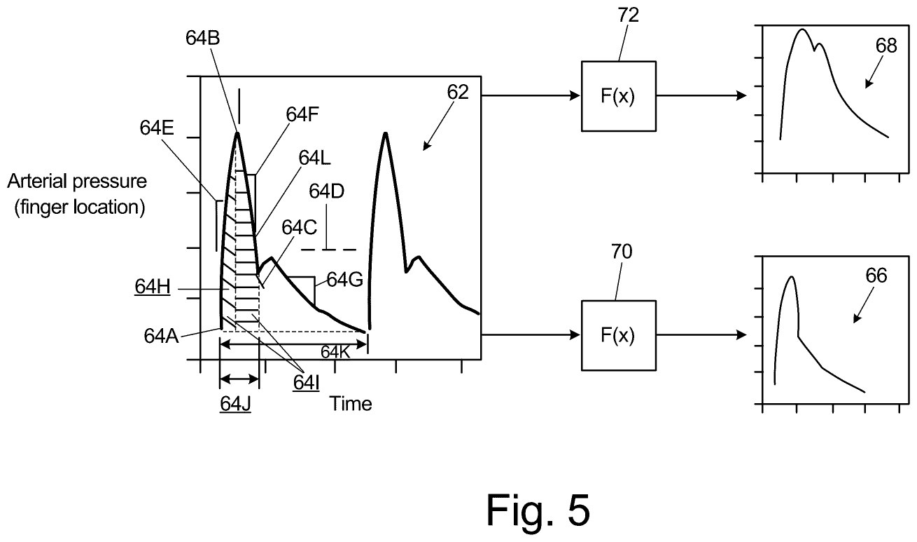

FIG. 5 is a schematic depicting features of exemplary arterial pressure waveform 62 taken at a patient's finger along with reconstructed brachial and radial arterial pressure waveforms derived from the arterial pressure waveform. Arterial pressure waveform 62 includes at least features 64A, 64B, 64C, 64D, 64E, 64F, 64G, 64H, 64I, 64J, 64K, and 64L, collectively arterial pressure features 64A-64L.

Features 64A, 64B, and 64C are the minimum arterial pressure (i.e., end diastolic pressure), maximum arterial pressure, and dichotic notch pressures, respectively, of a heartbeat. Feature 64D is the mean arterial pressure of a heartbeat (or series of heartbeats). Feature 64E is the maximum rate of pressure change during systolic rise. The maximum rate of pressure change during systolic decay is indicated by feature 64F. The maximum rate of change of the diastolic phase is represented by feature 64G. Feature 64H represents the area under arterial pressure waveform 62 between the minimum arterial pressure 64A and maximum arterial pressure 64B (i.e., during systolic rise) of a heartbeat, and feature 64I represents the area under arterial pressure waveform 62 between minimum arterial pressure 64A and dichotic pressure 64C (i.e., during the systolic phase). Feature 64J can be the time of the systolic phase for a given heartbeat. Feature 64K is the time between successive heartbeats of arterial pressure waveform 62 from which pulse rate can be determined. The exponential decay constant of the systolic phase is represented by feature 64L.

Additional features can be derived from arterial pressure features 64. For example, stroke volume is proportional to the systolic phase area (i.e., feature 64I) and, as such, can derived from systolic phase area (i.e., feature 64I). Cardiac output equals stroke volume multiplied by pulse rate (i.e., feature 64K). Systemic vascular resistance can be determined from the difference between mean arterial pressure and central venous pressure, divided by cardiac output.

Similar features can be extracted from radial arterial pressure waveform 66 and/or brachial arterial pressure waveform 68. Radial arterial pressure waveform 66 is derived from finger arterial pressure waveform 62 using transfer function 70. Brachial arterial pressure waveform 68 is derived from finger arterial pressure waveform 62 using transfer function 72. Each of transfer function 70 and transfer function 72 are determined by correlating arterial pressure features 64 of finger arterial pressure waveform 62 to corresponding features of radial arterial pressure waveform 66 and brachial arterial pressure waveform 68.

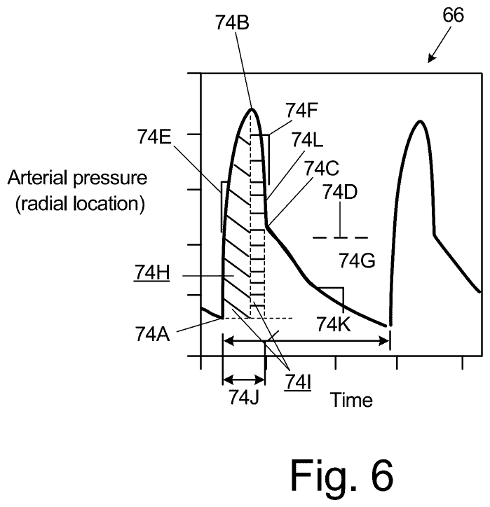

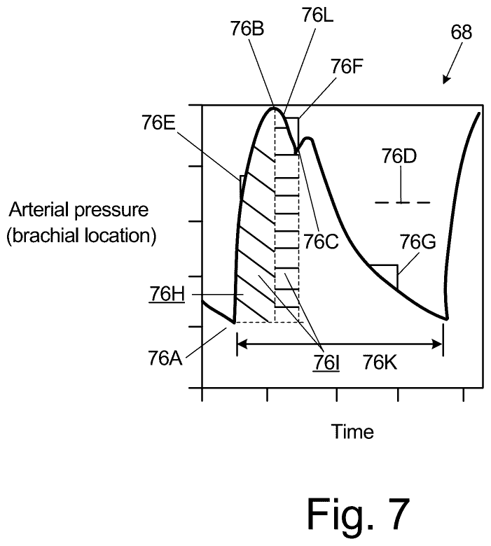

FIG. 6 is a chart depicting exemplary features 74A, 74B, 74C, 74D, 74E, 74F, 74G, 74H, 74I, 74K, and 74L, collectively radial arterial pressure features 74A-74L, of reconstructed radial pressure waveform 66. FIG. 7 is a chart depicting exemplary features 76A, 76B, 76C, 76D, 76E, 76F, 76G, 76H, 76I, 76K, and 76L, collectively brachial arterial pressure features 76A-76L, of reconstructed brachial pressure waveform 68. Each of features 74A-74L and features 76A-76L correspond to like features 64A-64L of finger pressure waveform 62 shown in FIG. 5.

Accordingly, corresponding features 74A-74L are determined from reconstructed radial arterial pressure waveform 66. Features 74A, 74B, and 74C are the minimum arterial pressure (i.e., end diastolic pressure), maximum arterial pressure, and dichotic notch pressures, respectively, of a heartbeat from reconstructed radial arterial pressure waveform 66. Feature 74D is the mean arterial pressure of a heartbeat (or series of heartbeats). Feature 74E is a maximum rate of pressure change during systolic rise. The maximum rate of pressure change during systolic decay is indicated by feature 74F. The maximum rate of change of the diastolic phase is represented by feature 74G. Feature 74H represents the area under radial arterial pressure waveform 66 between the minimum arterial pressure 74A and maximum arterial pressure 74B (i.e., during systolic rise) of a heartbeat, and feature 74I represents the area under radial arterial pressure waveform 66 between minimum arterial pressure 74A and dichotic pressure 74C (i.e., during the systolic phase). Feature 74J can be the time of the systolic phase for a given heartbeat. Feature 74K is the time between sequential heartbeats of reconstructed radial arterial pressure waveform 66 from which pulse rate can be determined. The exponential decay constant of the systolic phase is represented by feature 74L. Each of features 74A-74L is determined from radial arterial pressure waveform 66, which is reconstructed or derived from finger arterial pressure waveform 62.

Likewise, corresponding features 76A-76L are determined from brachial arterial pressure waveform 68. Features 76A, 76B, and 76C are the minimum arterial pressure (i.e., end diastolic pressure), maximum arterial pressure, and dichotic notch pressures, respectively, of a heartbeat from brachial arterial pressure waveform 68. Feature 76D is the mean arterial pressure of a heartbeat (or series of heartbeats). Feature 76E is a maximum rate of pressure change during systolic rise. The maximum rate of pressure change during systolic decay is indicated by feature 76F. The maximum rate of change of the diastolic phase is represented by feature 76G. Feature 76H represents the area under brachial arterial pressure waveform 68 between the minimum arterial pressure 76A and maximum arterial pressure 76B (i.e., during systolic rise) of a heartbeat, and feature 76I represents the area under brachial arterial pressure waveform 68 between minimum arterial pressure 76A and dichotic pressure 76C (i.e., during the systolic phase). Feature 76J can be the time of the systolic phase for a given heartbeat. Feature 76K is the time between sequential heartbeats of brachial arterial pressure waveform 68 from which pulse rate can be determined. The exponential decay constant of the systolic phase is represented by feature 76L. Each of features 76A-76L is determined from brachial arterial pressure waveform 68, which is reconstructed or derived from finger arterial pressure waveform 62.

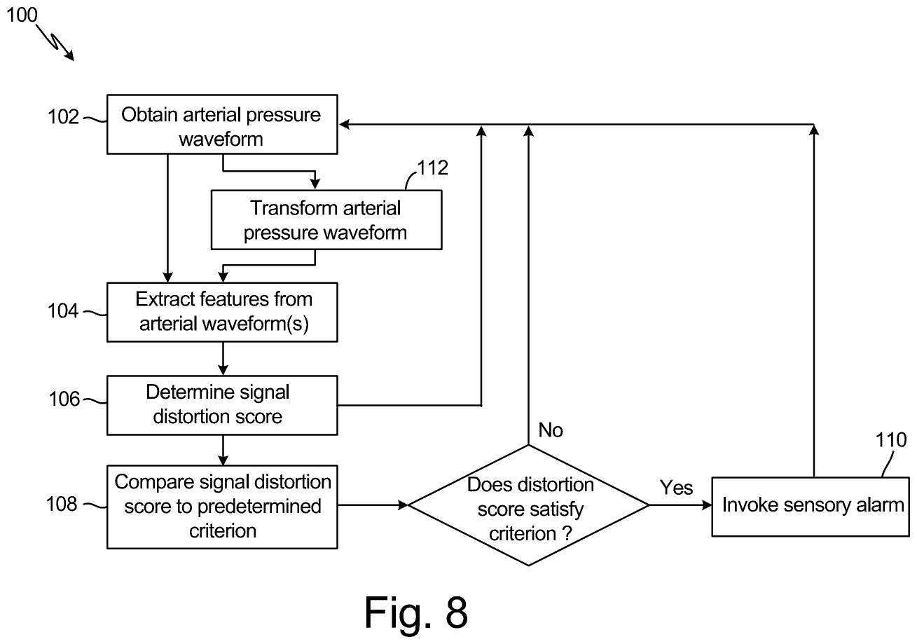

FIG. 8 is a flowchart describing signal distortion detection method 100 for evaluating the fit of finger cuff 42, or another hemodynamic sensor 12. Method 100 includes steps 102, 104, and 106. In further examples, method 100 can include one or more of steps 108, 110, and 112. The sequence depicted in FIG. 8 is for illustrative purposes only and is not meant to limit method 100 in any way as it is understood that the portions of method 100 can proceed in a different logical order, additional or intervening portions can be included, or described portions of method 100 can be divided into multiple portions, or described portions of method 100 can be omitted without detracting from the described above.

In step 102, an arterial pressure waveform is obtained from a patient monitoring system. For example, pressure controller 44 may vary a bladder pressure of finger cuff 42 in accordance with the volume clamp to obtain hemodynamic data representative of the patient's arterial pressure waveform. This hemodynamic data may be converted into digital hemodynamic data prior by analog-to-digital converter 27 located within pressure controller 44, hardware unit 16, or patient monitoring device 18. Based on the hemodynamic data, hardware unit 16 continuously determines an arterial pressure waveform (i.e., arterial pressure as a function of time). As generally represented by patient monitoring system 10, a different hemodynamic sensor 12 could be used that produces hemodynamic data representative of a patient's arterial pressure. In either example, step 102 includes measuring or calculating an arterial pressure waveform based on measurements of a hemodynamic sensor sensitive to fit and/or placement on the patient.

Step 104 includes extracting one or more features from the arterial pressure waveform measured or determined in step 102. As discussed above, features of the arterial pressure waveform can include one or more of mean arterial pressure, maximum arterial pressure, minimum arterial pressure, pulse rate, pulse pressure, maximum and/or minimum rate of pressure change with respect to time during a heartbeat (or a subset of heartbeats), maximum and/or minimum rate of pressure change with respect of time during the systolic phase and/or diastolic phase of a heartbeat (or a subset of heartbeats), end diastolic pressure, diastolic gradient, systolic pressure gradient, pulse transit time, cardiac output, stroke volume, blood temperature, systolic rise area, diastolic average pressure, systemic vascular resistance, exponential constant associated with systolic decay, among other possible features. Any one or more of the features can be extracted from finger arterial pressure waveform 62 or, as discussed further below, radial arterial pressure waveform 66 or brachial arterial pressure waveform 68, which are reconstructed or derived from finger arterial pressure waveform 62. In other examples, features can be extracted from each of the finger, radial, and brachial waveforms, or any two of the arterial waveforms (e.g., two arterial waveforms selected from finger arterial pressure waveform 62, radial arterial pressure waveform 66, and brachial arterial pressure waveform 68).

Step 106 includes determining distortion score 34 based on one or more of the extracted features. For example, a subset of the potential features can be incorporated into a distortion detection model using training methods discussed above. Continuing with this example, distortion coefficient associated with each extracted feature can be used to determine distortion score 34 based on Equation 1. In one example, distortion score 34 is determined based on one or more of average diastolic pressure, stroke volume, systolic vascular resistance of radial arterial pressure waveform 66 and systolic rise area and exponential decay constant of the systolic phase of brachial arterial waveform 68. Steps 102, 104, and 106 or steps 102, 104, 106, and 112 can be repeated to determine a trend of distortion score 34 during a period of time. In some examples, method 100 determines distortion score 34 as an average distortion score 34 over a period of time (e.g., a twenty-second time interval). In other examples, method 100 may determine whether distortion score 34 is increase or decreasing. In such examples, method 100 may determine a rate of increase or a rate of decrease of distortion score 34.

In step 108, distortion score 34 or a trend of distortion scores 34 (e.g., an average distortion score 34, rate of increasing distortion score 34, a rate of decreasing distortion score 34, or a combination of these distortion score trend parameters) is compared to a predetermined criterion or set of criteria. For example, distortion score 34 can be compared to one of multiple subranges of a nominal total range of distortion score 34. In a further example, distortion score 34 can be compared to a value of distortion score 34 and a trend of distortion score over a period time, the value of distortion score 34 associated with one or more subranges of a nominal range of distortion scores 34. In a further example, distortion score 34 can be a value within a nominal range divided into two consecutive subranges (e.g., a first subrange between 0 and 50 and a second range between 51 and 100 for a nominal range in which distortion score 34 can be a value between 0 and 100). For distortion scores 34 within the first subrange, hardware unit 16 may cause one or more of the following actions to occur: a) display distortion score 34 on user interface 36 of patient monitoring device 18 and b) display an indication (e.g., a message or other status indication) that hemodynamic sensor 12 is properly fit to patient 14. Alternatively, no message or status indication is provided when distortion score 34 indicates a proper fit and location of hemodynamic sensor 12. In this instance, steps 102, 104, and 106 can be repeated to provide continuous or periodic monitoring of hemodynamic sensor 12. For distortion scores 34 within the second subrange, hardware unit 16 can invoke sensory alarm in step 110. Thereafter, steps 102, 104, 106, 108, and 110 can be repeated continuously invoking sensory alarm until medical practitioner refits, repositions, and/or replaces hemodynamic sensor 12.

In some examples of method 100, the arterial pressure waveform obtained in step 102 can be mathematically transformed in step 112 to represent an arterial pressure waveform at a location different than the location of hemodynamic sensor 12. For example, arterial pressure waveforms produced by finger cuff 42 are representative of the arterial pressure within the patient's finger (or toe). The finger arterial pressure waveform can be transformed into an arterial pressure waveform at a radial location of the patient and/or a brachial location of the patient. Features of brachial and/or radial arterial pressure waveforms can be extracted in step 104 and a distortion score 34 determined in step 106 based on one or more features extracted from one or more of the finger arterial pressure waveform, the radial arterial pressure waveform, and the brachial arterial pressure waveform. Thereafter, the distortion score determined in accordance with step 100 can be compared to a predetermined criterion or criteria in step 108. Based on this comparison, the sensory alarm can be invoked in step 110. Whether or not sensory alarm is invoked, steps 102, 104, 106, 108, 110, and 112 are repeated to provide continuous and/or periodic monitoring of hemodynamic sensor 12 fit and/or location.

### Discussion of Possible Examples

The following are non-exclusive descriptions of possible examples of the present invention.

### System for Monitoring Hemodynamic Data of a Patient

A system for monitoring hemodynamic data of a patient according to an example of this disclosure includes, among other possible things, a hardware unit, a signal distortion detection code, a hemodynamic sensor, and a sensory alarm. The hardware unit includes a hardware processor, an analog-to-digital converter (ADC), and a system memory. The signal distortion detection code is stored in the system memory and includes a weighting module. The hemodynamic sensor is coupled to the hardware unit and fitted about an appendage of the patient. The hardware processor is configured to execute the signal distortion detection code to evaluate the fit of the hemodynamic sensor to the patient.

The system of the preceding paragraph can optionally include, additionally and/or alternatively, any one or more of the following features, configurations and/or additional components.

A further example of the foregoing system, wherein the hardware processor can be configured to execute the signal distortion detection code to obtain digital hemodynamic data converted, by the ADC, from a signal received from the hemodynamic sensor on an ongoing basis.

A further example of any of the foregoing systems, wherein the hardware processor can be configured to execute the signal distortion detection code to obtain a first arterial pressure waveform based on the digital hemodynamic data.

A further example of any of the foregoing systems, wherein the hardware processor can be configured to execute the signal distortion detection code to extract a plurality of features from the first arterial pressure waveform.

A further example of any of the foregoing systems, wherein the plurality of features can be indicative of a fit of the hemodynamic sensor to the patient.

A further example of any of the foregoing systems can include determining, using the weighting module, a distortion score corresponding to the likelihood that the hemodynamic sensor is misfit to the patient based on a weighted combination of the plurality of features.

A further example of any of the foregoing systems can include invoking the sensory alarm if the distortion score satisfies a predetermined distortion criterion.

A further example of any of the foregoing systems can include a patient monitoring device.

A further example of any of the foregoing systems, wherein the hardware unit can be housed within the patient control unit.

A further example of any of the foregoing systems, wherein the hemodynamic sensor can be a finger cuff that includes an inflatable bladder configured to wrap around finger of the patient, a light-emitting diode, and a photodiode spaced along a longitudinal dimension of the inflatable bladder from the light-emitting diode.

A further example of any of the foregoing systems, wherein the first arterial pressure waveform can be representative of an arterial pressure within the finger of the patient.

A further example of any of the foregoing systems can include a pressure control unit coupled to the finger cuff by a tube to receive the signal.

A further example of any of the foregoing systems, wherein the signal can be indicative of an air pressure within the inflatable bladder of the finger cuff.

A further example of any of the foregoing systems, wherein the pressure control unit can include a control valve, and a pump disposed along a conduit connecting the tube of the finger cuff to an intake port of the pressure control unit.

A further example of any of the foregoing systems, wherein the intake port of the pressure control unit can communicate with an ambient environment.

A further example of any of the foregoing systems, wherein the control valve of the pressure control unit can operate to vary the air pressure within the inflatable bladder based on a blood pressure of the patient.

A further example of any of the foregoing systems, wherein obtaining the first arterial pressure waveform includes transforming the digital hemodynamic data such that the first arterial pressure waveform is representative of one of a radial arterial pressure waveform and brachial arterial pressure waveform.

A further example of any of the foregoing systems, wherein the plurality of features can include at least one of an average diastolic pressure, a stroke volume, and a systemic vascular resistance derived from the first arterial pressure waveform representative of the radial arterial pressure waveform.

A further example of any of the foregoing systems, wherein the plurality of features can include at least one of a systolic rise area and an exponential decay constant of the systolic phase derived from the brachial arterial waveform.

A further example of any of the foregoing systems, wherein the plurality of features can include each of an average diastolic pressure, a stroke volume, and a systemic vascular resistance derived from the first arterial pressure waveform representative of the radial arterial pressure waveform and each of a systolic rise area and an exponential decay constant of the systolic phase derived a second arterial pressure waveform representative of the brachial arterial waveform.

A further example of any of the foregoing systems, wherein the plurality of features can include at least one of mean arterial pressure, maximum arterial pressure, minimum arterial pressure, pulse rate, pulse pressure, maximum rate of pressure change with respect to time, minimum rate of pressure change with respect to time, end diastolic pressure, diastolic gradient, systolic pressure gradient, pulse transit time, cardiac output, stroke volume, blood temperature, systolic rise area, diastolic average pressure, systemic vascular resistance, and exponential constant associated with systolic decay.

A further example of any of the foregoing systems, wherein at least one feature of the plurality of features can be extracted from a finger arterial pressure waveform, a radial arterial pressure waveform, or a brachial arterial pressure waveform.

A further example of any of the foregoing systems, wherein the predetermined distortion criterion can be based on at least one of a value of the distortion score and a trend of the distortion score over a time interval.

A further example of any of the foregoing systems, wherein the distortion score can equal a value within a nominal range of distortion scores subdivided into at least two subranges.

A further example of any of the foregoing systems, wherein the predetermined distortion criterion can be based on a value of the distortion score associated with a threshold between subranges.

A further example of any of the foregoing systems, wherein extracting the plurality of features from the first arterial pressure waveform can include extracting the plurality of features from a time interval starting with the application of the hemodynamic sensor to the patient or a physiological change of the patient.

A further example of any of the foregoing systems, wherein the time interval can be greater than or equal to twenty seconds and less than or equal to sixty seconds.

### Method for Use by a System for Monitoring Hemodynamic Data of a Patient

A method for use by any of the foregoing exemplary systems for monitoring hemodynamic data of the patient includes, among other possible steps, obtaining, by the signal distortion detection code executed by the hardware processor, digital hemodynamic data converted, by the ADC, from a signal received from the hemodynamic sensor on an ongoing basis. The method includes obtaining, by the signal distortion detection code executed by the hardware processor, a first arterial pressure waveform based on the digital hemodynamic data and extracting, by the signal distortion detection code executed by the hardware processor, a plurality of features from the first arterial pressure waveform. The plurality of features is indicative of distortion of the first arterial pressure waveform associated with a fit of the hemodynamic sensor to the patient. The method includes determining, by the signal distortion detection code using the weighting module and executed by the hardware processor, a distortion score corresponding to the likelihood that the hemodynamic sensor is misfit to the patient based on a weighted combination of the plurality of features. The method includes invoking, by the signal distortion detection code executed by the hardware processor, the sensory alarm if the distortion score satisfies a predetermined distortion criterion.

The method of the preceding paragraph can optionally include, additionally and/or alternatively, any one or more of the following features, configurations, additional components, and/or steps.

A further example of the foregoing method, wherein the predetermined distortion criterion can be based on at least one of a value of the distortion score and a trend of the distortion score over a time interval.

A further example of any of the foregoing methods, wherein obtaining the first arterial pressure waveform can include transforming, by the signal distortion detection code executed by the hardware processor, the digital hemodynamic data such that the first arterial pressure waveform can be representative of one of a radial arterial pressure waveform and a brachial arterial pressure waveform.

A further example of any of the foregoing methods, wherein the plurality of features can include at least one of an average diastolic pressure, a stroke volume, and a systemic vascular resistance, each feature derived from the first arterial pressure waveform representative of the radial arterial pressure waveform.

A further example of any of the foregoing methods can include obtaining, by the signal distortion detection code executed by the hardware processor, a second arterial pressure waveform based on the digital hemodynamic data.

A further example of any of the foregoing methods, wherein obtaining the second arterial pressure waveform can include transforming, by the signal distortion detection code executed by the hardware processor, the digital hemodynamic data such that the second arterial pressure waveform can be representative of the other of the radial arterial pressure waveform and the brachial arterial pressure waveform.

A further example of any of the foregoing methods, wherein the plurality of features can include at least one of a systolic rise area and an exponential decay constant of the systolic phase, each feature derived from the brachial arterial waveform.

A further example of any of the foregoing methods, wherein the plurality of features can include each of an average diastolic pressure, a stroke volume, and a systemic vascular resistance derived from the first arterial pressure waveform representative of the radial arterial pressure waveform and each of a systolic rise area and an exponential decay constant of the systolic phase derived from the second arterial waveform representative of the brachial arterial pressure waveform.

A further example of any of the foregoing methods, wherein the plurality of features can include at least one of mean arterial pressure, maximum arterial pressure, minimum arterial pressure, pulse rate, pulse pressure, maximum rate of pressure change with respect to time, minimum rate of pressure change with respect to time, end diastolic pressure, diastolic gradient, systolic pressure gradient, pulse transit time, cardiac output, stroke volume, blood temperature, systolic rise area, diastolic average pressure, systemic vascular resistance, and exponential constant associated with systolic decay.

A further example of any of the foregoing methods, wherein at least one feature of the plurality of features can be extracted from a finger arterial pressure waveform, a radial arterial pressure waveform, or a brachial arterial pressure waveform.

A further example of any of the foregoing methods, wherein the distortion score can equal a value within a nominal range of distortion scores subdivided into at least two subranges, and wherein the predetermined distortion criterion is based on a value of the distortion score associated with a threshold between subranges.

A further example of any of the foregoing methods, wherein obtaining digital hemodynamic data can include receiving a signal indicative of an arterial pressure waveform from a finger cuff.

A further example of any of the foregoing methods, wherein obtaining digital hemodynamic data can include controlling air pressure within a bladder of the finger cuff based on a blood pressure of the patient.

A further example of any of the foregoing methods, wherein the plurality of features can be extracted from a time interval starting with the application of the hemodynamic sensor to the patient or a physiological change of the patient.

A further example of any of the foregoing methods, wherein the time interval can be greater than or equal to twenty seconds and less than or equal to sixty seconds.

### A Computer-Readable Non-Transitory Medium

A computer-readable non-transitory medium according to an example of this disclosure includes, among other possible things, instructions stored thereon, which when executed by a hardware processor, initiate a method. The method includes obtaining a digital hemodynamic data converted, by an analog-to-digital converter (ADC), from a signal received from a hemodynamic sensor on an ongoing basis and obtaining a first arterial pressure waveform based on the digital hemodynamic data. The method includes extracting a plurality of features from the first arterial pressure waveform, each feature indicative of distortion of the first arterial pressure waveform associated with a fit of the hemodynamic sensor to a patient. The method includes determining a distortion score corresponding to the likelihood that the hemodynamic sensor is misfit to the patient based on a weighted combination of the plurality of features and invoking a sensory alarm if the distortion score satisfies a predetermined distortion criterion.

The computer-readable non-transitory medium of the preceding paragraph can optionally include, additionally and/or alternatively, any one or more of the following features, configurations, additional components, and/or instruction steps.

A further example of the foregoing computer-readable non-transitory medium, wherein the predetermined distortion criterion can be based on at least one of a value of the distortions score and a trend of the distortion score over a time interval.

A further example of any of the foregoing computer-readable non-transitory mediums, wherein the distortion score can equal a value within a nominal range of distortion scores subdivided into at least two subranges, and wherein the predetermined distortion criterion can be based on a value of the distortion score associated with a threshold between subranges.

A further example of any of the foregoing computer-readable non-transitory mediums, wherein obtaining the first arterial pressure waveform can include transforming the digital hemodynamic data such that the first arterial pressure waveform can be representative of one of a radial arterial pressure waveform and a brachial arterial pressure waveform.

A further example of any of the foregoing computer-readable non-transitory mediums, wherein the plurality of features can include at least one of an average diastolic pressure, a stroke volume, and a systemic vascular resistance derived from the first arterial pressure waveform representative of the radial arterial pressure waveform.

A further example of any of the foregoing computer-readable non-transitory mediums can include instructions, that when executed by the hardware processor, can obtain a second arterial pressure waveform based on the digital hemodynamic data.

A further example of any of the foregoing computer-readable non-transitory mediums, wherein obtaining the second arterial pressure waveform can include transforming the digital hemodynamic data such that the second arterial pressure waveform can be representative of the other of the radial arterial pressure waveform and the brachial arterial pressure waveform.

A further example of any of the foregoing computer-readable non-transitory mediums, wherein the plurality of features can include at least one of a systolic rise area and an exponential decay constant of the systolic phase derived from the second arterial pressure waveform representative of the brachial arterial waveform.

A further example of any of the foregoing computer-readable non-transitory mediums, wherein the plurality of features can include each of an average diastolic pressure, a stroke volume, and a systemic vascular resistance derived from the first arterial pressure waveform representative of the radial arterial pressure waveform and each of a systolic rise area and an exponential decay constant of the systolic phase derived from the brachial arterial waveform.

A further example of any of the foregoing computer-readable non-transitory mediums, wherein the plurality of features can include at least one of mean arterial pressure, maximum arterial pressure, minimum arterial pressure, pulse rate, pulse pressure, maximum rate of pressure change with respect to time, minimum rate of pressure change with respect to time, end diastolic pressure, diastolic gradient, systolic pressure gradient, pulse transit time, cardiac output, stroke volume, blood temperature, systolic rise area, diastolic average pressure, systemic vascular resistance, and exponential constant associated with systolic decay.

A further example of any of the foregoing computer-readable non-transitory mediums, wherein the first arterial waveform can be representative of a finger arterial pressure waveform, a radial arterial pressure waveform, or a brachial arterial pressure waveform.

A further example of any of the foregoing computer-readable non-transitory mediums, wherein the second arterial waveform can be representative of a finger arterial pressure waveform, a radial arterial pressure waveform, or a brachial arterial pressure waveform.

A further example of any of the foregoing computer-readable non-transitory mediums, wherein the plurality of features can be extracted from a time interval starting with the application of the hemodynamic sensor to the patient or a physiological change of the patient.

A further example of any of the foregoing computer-readable non-transitory mediums, wherein the time interval can be greater than or equal to twenty seconds and less than or equal to sixty seconds.

While the invention has been described with reference to an exemplary example(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular example(s) disclosed, but that the invention will include all examples falling within the scope of the appended claims.

1. A system for monitoring hemodynamic data of a patient, the system comprising:
a hardware unit including a hardware processor, an analog-to-digital converter (ADC), and a system memory;
a signal distortion detection code stored in the system memory and including a weighting module;
a hemodynamic sensor coupled to the hardware unit and fitted about an appendage of the patient; and
a sensory alarm;
wherein the hardware processor is configured to execute the signal distortion detection code to:
obtain digital hemodynamic data converted, by the ADC, from a signal received from the hemodynamic sensor on an ongoing basis;
obtain a first arterial pressure waveform based on the digital hemodynamic data;
extract a plurality of features from the first arterial pressure waveform, wherein the plurality of features is indicative of a fit of the hemodynamic sensor to the patient;
determine, using the weighting module, a distortion score corresponding to the likelihood that the hemodynamic sensor is misfit to the patient based on a weighted combination of the plurality of features; and
invoke the sensory alarm if the distortion score satisfies a predetermined distortion criterion.

2. The system of claim 1, wherein the hemodynamic sensor is a finger cuff comprising:
an inflatable bladder configured to wrap around a finger of the patient;
a light-emitting diode; and
a photodiode spaced along a longitudinal dimension of the inflatable bladder from the light-emitting diode;
wherein the first arterial pressure waveform is representative of an arterial pressure within the finger of the patient.

3. The system of claim 2, further comprising:
a pressure control unit coupled to the finger cuff by a tube to receive the signal, wherein the signal is representative of an air pressure within the inflatable bladder.

4. The system of claim 3, wherein the pressure control unit comprises a control valve and a pump disposed along a conduit connecting the tube to an intake port in communication with an ambient environment, and wherein the control valve is operable to vary the air pressure within the inflatable bladder based on a blood pressure of the patient.

5. The system of claim 3, further comprising:
a patient monitoring device, wherein the hardware unit is housed within the pressure control unit or attached to the pressure control unit.

6. The system of claim 1, wherein obtaining the first arterial pressure waveform includes:
transforming the digital hemodynamic data such that the first arterial pressure waveform is representative of one of a radial arterial pressure waveform and a brachial arterial pressure waveform.

7. The system of claim 6, wherein the plurality of features comprises at least one of an average diastolic pressure, a stroke volume, and a systemic vascular resistance derived from the first arterial pressure waveform representative of the radial arterial pressure waveform.

8. The system of claim 7, wherein the hardware processor is configured to execute the signal distortion detection code to:
obtain a second arterial pressure waveform based on the digital hemodynamic data, wherein obtaining the second arterial pressure waveform includes:
transforming the digital hemodynamic data such that the second arterial pressure waveform is representative of the other of the radial arterial pressure waveform and the brachial arterial pressure waveform.

9. The system of claim 8, wherein the plurality of features comprises at least one of a systolic rise area and an exponential decay constant of a systolic phase, each feature derived from the brachial arterial waveform.

10. The system of claim 9, wherein the plurality of features includes each of an average diastolic pressure, a stroke volume, and a systemic vascular resistance derived from the first arterial pressure waveform representative of the radial arterial pressure waveform and each of a systolic rise area and an exponential decay constant of a systolic phase derived from the second arterial pressure waveform representative of the brachial arterial pressure waveform.

11. The system of claim 1, wherein the plurality of features includes at least one of mean arterial pressure, maximum arterial pressure, minimum arterial pressure, pulse rate, pulse pressure, maximum rate of pressure change with respect to time, minimum rate of pressure change with respect to time, end diastolic pressure, diastolic gradient, systolic pressure gradient, pulse transit time, cardiac output, stroke volume, blood temperature, systolic rise area, diastolic average pressure, systemic vascular resistance, and exponential constant associated with systolic decay.

12. The system of claim 11, wherein at least one feature of the plurality of features is extracted from a finger arterial pressure waveform, a radial arterial pressure waveform, or a brachial arterial pressure waveform.

13. The system of claim 1, wherein the predetermined distortion criterion is based on at least one of a value of the distortion score and a trend of the distortion score over a time interval.

14. The system of claim 1, wherein the distortion score equals a value within a nominal range of distortion scores subdivided into at least two subranges, and wherein the value of the distortion score is associated with a threshold between subranges.

15. The system of claim 1, wherein extracting the plurality of features from the first arterial pressure waveform includes extracting the plurality of features from a time interval starting with application of the hemodynamic sensor to the patient or physiological change of the patient.

16. The system of claim 15, wherein the time interval is greater than or equal to twenty seconds and less than or equal to sixty seconds.

17. A method for use by a system for monitoring hemodynamic data of a patient, the system comprising a hemodynamic sensor, a sensory alarm, and a hardware unit including a hardware processor, an analog-to-digital converter (ADC), and a signal distortion detection code stored in a system memory and including a weighting module, the method comprising:
obtaining, by the signal distortion detection code executed by the hardware processor, digital hemodynamic data converted, by the ADC, from a signal received from the hemodynamic sensor on an ongoing basis;
obtaining, by the signal distortion detection code executed by the hardware processor, a first arterial pressure waveform based on the digital hemodynamic data;
extracting, by the signal distortion detection code executed by the hardware processor, a plurality of features from the first arterial pressure waveform indicative of distortion of the first arterial pressure waveform associated with a fit of the hemodynamic sensor to the patient;
determining, by the signal distortion detection code using the weighting module and executed by the hardware processor, a distortion score corresponding to the likelihood that the hemodynamic sensor is misfit to the patient based on a weighted combination of the plurality of features; and
invoking, by the signal distortion detection code executed by the hardware processor, the sensory alarm if the distortion score satisfies a predetermined distortion criterion.

18. The method of claim 17,
wherein the predetermined distortion criterion is based on at least one of a value of the distortion score and a trend of the distortion score over a time interval; and
wherein obtaining the first arterial pressure waveform includes:
transforming, by the signal distortion detection code executed by the hardware processor, the digital hemodynamic data such that the first arterial pressure waveform is representative of one of a radial arterial pressure waveform and a brachial arterial pressure waveform.

19. The method of claim 18, wherein the plurality of features comprises at least one of an average diastolic pressure, a stroke volume, and a systemic vascular resistance, each feature derived from the first arterial pressure waveform representative of the radial arterial pressure waveform.

20. A computer-readable non-transitory medium having stored thereon instructions, which when executed by a hardware processor, initiate a method comprising:
obtaining a digital hemodynamic data converted, by an analog-to-digital converter (ADC), from a signal received from a hemodynamic sensor on an ongoing basis;
obtaining a first arterial pressure waveform based on the digital hemodynamic data;
extracting a plurality of features from the first arterial pressure waveform, each feature indicative of distortion of the first arterial pressure waveform associated with a fit of the hemodynamic sensor to a patient;
determining a distortion score corresponding to the likelihood that the hemodynamic sensor is misfit to the patient based on a weighted combination of the plurality of features; and
invoking a sensory alarm if the distortion score satisfies a predetermined distortion criterion.
